(19) United States
(12) Patent Application Publication
(10) Pub. No.: US 2011/0091081 A1
(43) Pub. Date: Apr. 21, 2011

(54) METHOD AND SYSTEM FOR ANALYZING THE EXPRESSION OF BIOMARKERS IN CELLS IN SITU IN THEIR TISSUE OF ORIGIN

(75) Inventors: Brion Daryl Sarachan, Schenectady, NY (US); Thomas Paul Repoff, Sprakers, NY (US); Colin Craig McCulloch, Charlton, NY (US); Fiona Mary Ginty, Saratoga Springs, NY (US); Megan Pearl Rothney, Saratoga Springs, NY (US); Zhengyu Pang, Clifton Park, NY (US)

(73) Assignee: General Electric Company, Schenectady, NY (US)

(21) Appl. No.: 12/580,651

(22) Filed: Oct. 16, 2009

(51) Int. Cl. G06K 9/00; C12Q 1/02; C12Q 1/68; G06F 19/00

(52) U.S. Cl. 382/128; 435/29; 435/6; 702/19

(57) ABSTRACT

The present application discloses a technique for obtaining and storing data on expression of multiple biomarkers in individual cells or the compartments of individual cells in a tissue specimen and methods of utilizing that data to create groups, the members of which share some degree of similarity greater than the general population from which the data is drawn, by an analysis of digital images of a portion of the tissue specimen which has been iteratively stained to generate optical signals, typically fluorescent, reflective of the amount of each of the biomarkers examined. The analysis of the images involves a segmentation routine whereby each pixel of the examined images is assigned to an individual cell or a compartment of an individual cell, the intensity of the signal representative of each biomarker is measured for each pixel, a dataset is created in which each cell or compartments of each cell is associated with a signal intensity for each biomarker examined, and the dataset is interrogated with appropriate numerical tools to create groups. It also discloses the display of such groups on images of the tissue examined such that the individual cells belonging to a particular group are marked or indicated on one of the images examined. It further discloses examining the biomarker data for each group for possible association with a biological condition or process in cases in which tissue specimens drawn from different subjects or different portions of the tissue of a subject have been examined. It also discloses using this examination in the diagnoses, prognoses or assessment of the response to therapy of a condition or disease.

### BACKGROUND

The invention relates generally to a method of analyzing and visualizing the expression of biomarkers in individual cells wherein the cells are examined in situ in their tissue of origin to develop patterns of expression by numerical evaluation and a system to perform this analysis.

The examination of tissue specimens that have been treated to reveal the expression of biomarkers has long been a valuable tool for biological research and clinical studies. A common treatment has involved the use of antibodies or antibody surrogates such as antibody fragments that are specific for the biomarkers, commonly proteins, of interest. It is typical to directly or indirectly label such antibodies or antibody surrogates with a moiety capable, under appropriate conditions, of generating a signal. One approach has been to attach a fluorescent moiety to the antibody and to interrogate the treated tissue for fluorescence. The signal obtained is commonly indicative of not only the presence but also the amount of biomarker present.

The techniques of tissue treatment and examination have been refined so that the level of expression of a given biomarker in a particular cell or even a compartment of the given cell such as the nucleus, cytoplasm or membrane can be quantitatively determined. Typically the boundaries of these compartments or the cell as a whole are located using well-known histological stains. Commonly the treated tissue is examined with digital imaging and the level of different signals emanating from different biomarkers can consequently be readily quantitated.

More recently a technique has been developed which allows testing a given tissue specimen for the expression of numerous biomarkers. Generally this technique involves staining the specimen with a fluorophore labeled probe to generate signal for one or more probe bound biomarkers, chemically bleaching these signals and re-staining the specimen to generate signals for some further biomarkers. The chemical bleaching step is convenient because there are only a limited number of signals that can be readily differentiated from each other so only a limited number of biomarkers can be examined in a particular step. But with bleaching, the sample may be re-probed and re-evaluated for multiple steps. This cycling method may be used on formalin fixed paraffin embedded tissue (FFPE) samples and cells. Digital images of the specimen are collected after each staining step. The successive images of such a specimen can conveniently be kept in registry using morphological features such as DAPI stained cell nuclei, the signal of which is not modified by the chemical bleaching method.

Another approach has been to examine frozen tissue specimens by staining them iteratively and photo bleaching the labels from the previous staining step before applying the next set of stains. The strength of the fluorescent signal associated with each biomarker evaluated is then extracted from the appropriate image.

There have been efforts to utilize this data to identify patterns of biomarker expression. One approach has been to look for such patterns in an entire tissue specimen and to binarize the fluorophore signals using a threshold values and generate various expression profiles that are then overlaid on an image of the tissue of interest.

### BRIEF DESCRIPTION

The present invention involves the measurement of the level of expression of multiple biomarkers in individual cells or in the subcellular compartments of the individual cells in situ in the tissue of origin of the cells. The cells are conveniently segmented into individual cell units and their subcellular compartments (including membrane, cytoplasm and nucleus) as part of the data acquisition. The measurements associated with each cell or compartment is stored in a database and the database is interrogated with numerical methods to group cells together based on the similarity of their pattern of biomarker expression. The measurements are conveniently made by treating tissue specimens so that they can be examined for optical signals representative of the biomarker expression levels, the optical signals associated with the individual cells or their compartments and the optical signals captured by digital camera. The database typically preserves the original measurement values and the location, cell or compartment of the cell, from which each measurement is drawn. The numerical methods used to interrogate the database typically involves a computer algorithm which iteratively groups cells together based upon the similarity of their pattern of biomarker expression with the zero level of iteration assigning each cell to its own group and the final level of iteration assigning all the cells to a single group. Numerical methods that may be used include a rule based analysis, a classical statistical analysis and a learning algorithm such as a neural network.

The measurements may be performed on a single tissue specimen or on multiple specimens that are drawn from a single subject or multiple subjects. In one embodiment a single tissue specimen is examined to determine what patterns of biomarker expression it may display. In another embodiment tissue specimens from different locations in a particular subject are examined to determine how they differ in patterns of biomarker expression. For instance neoplastic tissue and normal tissue from the same organ of an subject may be examined to find any differences in their patterns of biomarker expression. In yet another embodiment specimens of similar tissue from different subjects may be examined. For instance tumor tissue from subjects treated with a cancer drug under evaluation and control subjects (perhaps subjects treated with placebo) may be examined to determine if the treatment has had any effect on the patterns of biomarker expression.

The measurements may be conveniently made by treating the tissue specimens under examination with labeled antibodies or antibody surrogates such as epitope specific antibody fragments and measuring the amount of each label that is bound. The antibodies or antibody surrogates are specific for the biomarkers of interest and are typically directly or indirectly labeled with moieties that give off optical signals when interrogated with light of the appropriate wavelength. In one embodiment the tissue specimens are repeatedly treated, with each treatment involving antibodies or antibody surrogates specific to different biomarkers than those involved in any other treatment and the signal generation from the immediately previous treatment is neutralized by optical or chemical means. The amount of each label bound to the biomarkers of interest by the antibodies or antibody surrogates may be conveniently measured by subjecting the specimen to light of the appropriate wavelength and digitally imaging the response.

The measurement values are conveniently preserved in a database which also preserves the identity of the measurement including the tissue and the location within the tissue from which it was drawn. The location should include the particular cell from which a particular measurement was drawn and may also include the compartment, nucleus, cytoplasm or membrane, associated with the measurement. The measurement values may be normalized using any convenient statistical technique. One convenient approach is to determine the mean and standard deviation of all the measurements associated with a given biomarker in a given study and subtract this mean value from each measurement value and then to divide the resultant difference by the standard deviation. This normalized or standardized value may be stored in the database or generated as part of the numerical interrogation of the database.

The numerical methods used to interrogate the database involve assigning certain attributes to each cell of interest based upon the measurements of biomarker expression levels and grouping those cells together which have similar biomarker expression attributes. The grouping typically uses an algorithm which groups together those cells which have a minimum distance between them in attribute space, i.e. two cells are included in the same group based on their distance from each other in n-dimensional space wherein each attribute is assigned a dimension. In one embodiment the attributes are simply the expression levels of the biomarkers of interest. In another embodiment the attributes include interrelationships between some of the biomarker expression levels. For instance, one attribute may be the ratio of the level of expression of a given biomarker in a cell's nucleus to that in its membrane or the level of expression of a given biomarker in a cell's cytoplasm compared to that in its membrane. Another attribute may be the ratio of the level of expression in a given cell of one biomarker to that of another. For example this may be the ratio between a protein and a phosphorylated version of the same protein. Typically the grouping proceeds iteratively initially grouping together only the cells that are the closest to each other in attribute space and then relaxing the similarity criterion until all the cells of interest have been included in a single group.

At any level of similarity a profile may be developed for each of the groups. Such a profile may typically comprise the mean or median value for that group of each attribute included in creating the grouping. For instance, if a grouping is based upon the level of expression of 10 biomarkers the profile for any given group may be the median value for that group of the level of expression of each of those 10 biomarkers. If the grouping were also based upon some ratios the profile would also include the median value for the group of each of those ratios.

The groupings may be conveniently visualized by an overlay over one or more of the digital images utilized to make the measurements of the levels of expression of the biomarkers. The overlay may conveniently show where in the original image cells occur which possess the profile possessed by a given group. Images from different tissue specimens with such overlays may be compared to determine if the patterns of cells with one or more profiles, i.e. patterns of cells which belong to one or more groups, are indicative of any biological condition or process. For instance a clinical study could be undertaken to establish an association between a given pattern and a certain diagnosis or prognosis such as survival time for a certain type of cancer.

The groupings may also be a convenient basis for investigating associations between a biological condition and a given cell attribute. Each grouping may be conveniently examined to identify any cell attribute which is associated with the diagnoses or prognoses of a given condition or disease or with the response to a given therapy for a given condition or disease. For instance, in a particular cell group it may be that the level of expression of a given biomarker is associated with a response to a given therapy, say administration of a drug, even though the level of expression for that biomarker in the tissue in general does not display such an association.

Another approach to using the data acquired from an examination of individual cells or the compartments of individual cells on the pattern of expression of biomarkers is to create groups of subjects for current or future association with a biological condition or process. In this application the same tissue in numerous subjects is examined using a panel of biomarker probes and the results for each pixel of the examined images is recorded and each pixel is associated with an individual cell or the compartment of an individual cell using a segmentation routine. Then the subjects may be placed into groups based on how similar that subject's expression of the biomarkers is to that of other members of the group. The comparison is based upon a summary of the biomarker expression data for each subject summarized at the cell level or the cell compartment level. For instance, one could gather breast cancer tumor tissue from a number of subjects and then group the subjects depending on the biomarker expression pattern of each subject wherein the pattern is built up from an examination of the individual cells or their compartments as opposed to a pattern built up from a tissue specimen without regard to segmentation into cells.

The availability of the database allows iterative and interactive investigations of how the biomarker groupings change in context of different biological questions. For example, all cells that are positive for Glut1, indicative of hypoxia, may be highlighted and expression of biomarkers associated with those positive cells may be visualized. This is particularly useful for quality control assessments and investigations of new hypotheses.

The groupings may also be conveniently used to guide a numerical analysis to identify one or more biomarkers whose levels of expression in a given group of cells is indicative of the presence, prognoses or response to treatment of a condition or disease. In one embodiment biomarker expression level data is available for cells from tissue of one or more control subjects and from tissue of one or more subjects with the condition or disease or subjected to a treatment of interest. This data is then examined to identify any associations in the level of expression of any biomarker in any given group between cells from the control subjects and cells from the target group.

### DRAWINGS

These and other features, aspects, and advantages of the present invention will become better understood when the following detailed description is read with reference to the accompanying drawings in which like characters represent like parts throughout the drawings, wherein:

FIG. 1 is a digital image at 20× magnification of xenograft tumor tissue of human colon cancer implanted in a mouse in which those cells belonging to Group 1 of the three group analysis outlined in Table 2 have been marked with a star.

FIG. 2 is the same base image as FIG. 1 but in this case with the cells belonging to Group 2 of the three group analysis outlined in Table 2 marked with a square.

FIG. 3 is the same base image as FIG. 1 but in this case with the cells belonging to Group 3 of the three group analysis outlined in Table 2 marked with a diamond.

FIG. 4 is the same base image as FIG. 1 but in this case the cells have been overlaid with markers for all three groups of the three group analysis outlined in Table 2 with Group 1 marked with a star, Group 2 marked with a square and Group 3 marked with a diamond.

FIG. 5 is similar to FIG. 1 but in this case the cells belonging to Group 1 of the three group analysis outlined in Table 2 have been shaded in.

FIG. 6 is similar to FIG. 2 but in this case the cells belonging to Group 2 of the three group analysis outlined in Table 2 have been shaded in.

FIG. 7 is similar to FIG. 3 but in this case the cells belonging to Group 3 of the three group analysis outlined in Table 2 have been shaded in.

### DETAILED DESCRIPTION

The present invention involves capturing data on the expression of biomarkers within the compartments of individual cells located within their tissue of origin, preserving this data on a cell by cell basis, analyzing this data to reveal patterns of expression, creating subsets of cells based on these patterns, visualizing the occurrence of these subsets on images of the tissues of origin and analyzing the occurrence of certain biomarkers in the subsets of cells for association to the diagnoses or prognoses of a condition or disease or to the response to treatment. The data can conveniently be initially captured by the treatment and imaging of tissue specimens. The treatment typically involves preparing slides of the tissue specimens and appropriately staining them to identify cell boundaries, cell compartment boundaries and levels of expression of selected biomarkers. The imaging typically involves digital imaging of selected fields of view from microscopic examination of the slides of the tissue specimens in a manner that the same field of view can be imaged after successive rounds of staining and the successive images can be placed in registry. The imaging also typically involves a segmentation routine that allows each pixel examined to be associated with a particular cell and a particular compartment of that cell. The data from this imaging is conveniently stored in a database such that each cell examined is associated with certain attributes reflective of the expression of the selected biomarkers within that cell. This database is then typically interrogated with numerical tools to group together those cells that have similar patterns of biomarker expression with the tools being able to create various size groups based on how similar the members of each group are to each other. One or more of these groupings can be conveniently visualized by an overlay of one or more markers or indicators on images of the tissue of origin of a given set of cells. In one embodiment an image of a selected field of view of a given slide is generated on which are marked all the cells which belong to a given group created by the application of the numerical tools. In one embodiment the pattern of biomarker expression within any given group of cells is analyzed for associations to the source of the tissue. In this embodiment tissue specimens are taken from at least two distinct groups of the same organism for instance an animal model or human subjects that differ in a biological feature under examination.

The techniques of the present invention can be applied to any tissue that is likely to vary in some manner as a result of its biological condition or history. For instance, the technique can be applied to the diagnoses of a condition by obtaining appropriate tissue specimens from subjects with and without a particular condition or disease. Thus one could take breast tissue or prostate tissue if the object were to diagnose breast or prostate cancer. Alternatively it could be applied to the prognoses of a disease or condition using appropriate historical tissue from subjects whose later clinical outcomes were known. Thus the techniques of the present invention could be applied to try to improve the prediction of survival rates in colon cancer patients from that available from the ratio of cMET expression in cytoplasm to that in membrane in which the ratio is based upon all the cells in the examined tissue. Additionally the techniques of the present invention could be applied to assess the effects of various treatments on a disease or condition. Thus one could use it to compare tumor tissue from untreated model animals to tumor tissue from model animals treated with one or more cancer drugs.

The biomarkers used in practicing the present invention may be any which are accessible to a histological examination that will give some indication of their level of occurrence or expression and are likely to vary in response to the biological condition or history of a selected tissue. The biomarkers may be DNA, RNA or protein based or a combination of them. Thus one could investigate whether there was a pattern of cells within a tissue with a given gene having a certain level of occurrence different from the average level of occurrence among all the cells in that tissue. One could similarly investigate for patterns of cells having a different level of RNA or protein expression.

The biomarkers may be conveniently selected in accordance with the biological phenomenon being examined Thus for instance if a particular biological pathway were involved in the phenomenon under examination proteins involved in that pathway or the RNA encoding those proteins could be selected as the biomarkers. For instance, if the proliferation of neoplastic tissue were the focus the Ki67 protein marker of cell proliferation could be selected. On the other hand if the focus were on hypoxia the Glu1 protein marker could be selected.

The level of expression of a biomarker of interest is conveniently assessed by staining the slides of the tissue with a probe specific to the biomarker associated with a label that can generate a signal under appropriate conditions. Two useful probes are DNA probes with sequences complimentary to the DNA or RNA of interest and antibodies or antibody surrogates such as antibody fragments with epitope specific regions that specifically bind to the biomarker of interest that may be DNA, RNA or protein. It is important that the probe be labeled in such a manner that the strength of the signal obtained from the label is representative of the amount of probe which has bound to its target.

A convenient probe from the point of view of availability and well established characterization is a monoclonal or polyclonal antibody specific for the biomarker of interest. There are commercially available antibodies specific to a wide variety of biomarkers. Mechanisms for associating many of these antibodies with labels are well established. In many cases the binding behavior of these antibodies is also well established.

A convenient label for the biomarker probes is a moiety that gives off an optical signal. A particularly convenient label is a moiety that gives off light of a defined wavelength when interrogated by light of an appropriate wavelength such as a fluorescent dye. Preferred fluorescent dyes are those that can be readily chemically conjugated to antibodies without substantially adversely affecting the ability of the antibodies to bind their targets.

A convenient approach for labeling if numerous biomarkers are to be examined is to directly label the antibodies. While there are sometimes certain advantages in using secondary or tertiary labeling like using an unlabeled primary antibody and a labeled secondary antibody against the species of the primary antibody such as signal amplification complications may arise in finding sufficient different systems for multiple rounds of staining and bleaching.

The slides are conveniently stained with the labeled biomarker probes using well established cytology procedures. The initial staining of each slide may also involve the use of markers for one or more of the cell compartments of nucleus, cytoplasm and membrane. It is convenient to use markers such as DAPI that are not bleached when the labels attached to the biomarker probes are bleached. These procedures generally involve rendering the biomarkers in the slide tissue accessible to the labeled probes and incubating the labeled probes with the so prepared slides for an appropriate period of time. The slides can be simultaneously incubated with a number of labeled biomarker probes, each specific for a different biomarker. However, there is a practical limit to the number of labeled probes that can be simultaneously incubated with a slide because each labeled probe must generate a signal which is fairly distinguishable from the signals from the other labeled probes. A convenient approach to staining numerous biomarkers is to stain a limited number of biomarkers, take appropriate images of the stained slide and then optically or chemically bleach the labels to destroy their ability to generate signal. A further set of labeled probes specific to different biomarkers but with labeling moieties identical to those used in the prior staining step can then be used to stain the same slide. This approach can be used iteratively until images have been acquired of the same slide stained for all the biomarkers of interest. One way of implementing such an approach is set forth in U.S. Published Patent Application 2008-0118934, incorporated herein by reference.

If more than one image is taken of a given field of view it is important that the successive images, commonly collectively referred to as a stack, be kept in registry. Thus if the approach of iteratively staining and bleaching a slide is used to obtain information on numerous biomarkers it is necessary to provide a mechanism for the images of each field of view from each round to be properly aligned with the images of the same field of view from previous rounds. A convenient approach is to ensure the presence of the same feature or features in each image of a field of view. One such feature that is particularly convenient is the pattern of cell nuclei as revealed by an appropriate stain such as DAPI. One of the images can then be taken as a reference, typically the first image taken, and appropriate transformations can be applied to the other images in that stack to bring them into registry. A technique for bringing images of the same field of view into registry with each other based on their cell nuclei pattern is disclosed in U.S. Published Patent Application 2008/0032328 incorporated herein by reference.

A representative number of fields of view are typically selected for each tissue sample depending upon the nature of the sample. For instance if a slide has been has been made of a single tissue specimen numerous fields of view may be available while if the target of examination is a tissue microarray (TMA) a more limited number of fields of view may be practical.

The images of each field of view are conveniently made with a digital camera coupled with an appropriate microscope and appropriate quality control routines. For instance the microscope may be designed to capture fluorescent images and be equipped with appropriate filters as well as being controlled by software that assures proper focus and correction for auto-fluorescence. One such routine for auto-fluorescence involves taking a reference image using the filter appropriate for a given fluorescent label but with no such label active in the image and then using this reference image to subtract the auto-fluorescence at that wavelength window from an image in which the fluorescent label is active.

Each image of each field of view may then be examined for segmentation into cells and the cellular compartments of nucleus, cytoplasm and membrane, and other cellular compartments. This segmentation is typically aided by the presence of stains from markers for these three compartments. As part of the segmentation procedure each pixel of each image is associated with a particular cell and a compartment of that cell. Then a value for the level of expression of each biomarker of interest is associated with each pixel from the level of signal from that pixel of the label for that biomarker. For instance if the label associated with the FOXO3a probe was Cy3, the pixels of the image of a given field of view that were stained with the labeled probe for FOXO3a would be evaluated for the fluorescent signals they exhibited in the wavelength window for Cy3. These values would then be associated with that biomarker for each of the pixels.

A database is conveniently created in which each compartment of each cell examined is associated with a value for each biomarker evaluated which reflects the strength of the signal from the label associated with the probe for that biomarker for all the pixels associated with that compartment. Thus a sum is taken across all the pixels associated with a given compartment of a given cell for the signal strength associated with each biomarker evaluated.

The database may be subject to a quality control routine to eliminate cells of compromised analytic value. For instance all the cells that do not lie wholly within the field of view and any cells that do not have between 1 and 2 nuclei, a membrane and a certain area of cytoplasm may be eliminated. This typically results in the elimination of between about 25% and 30% of the data.

The remaining data in the database may now be conveniently transformed and interrogated. The data for a given biomarker across all the cells examined may not follow a distribution which readily lends itself to standard statistical treatment. Therefore it may be useful to subject it to a transformation such as a Box Cox transformation that preserves the relative rankings of the values associated with a given biomarker but places such values into an approximate Normal distribution. Then it may be helpful to standardize the values associated with each biomarker so that the values for all the biomarkers have a common base. One approach is to determine the mean value and standard distribution of all the transformed values associated with a given biomarker and then to subtract this mean value from each value in the set for that biomarker and divide the difference by the standard deviation for that transformed dataset. The database may now be interrogated for groups of cells that have similar profiles of biomarker expression.

The data on biomarker expression levels in the database may be further transformed by creating three or more intervals of value and assigning a single value to each entry that falls within a given interval. This will make the biomarker expression level a semi-continuous variable. This may be useful for reducing the computational capacity needed for the grouping algorithm, especially for particularly large datasets.

The database may be interrogated with numerical tools to group together cells with some similarity in their expression of the biomarkers being examined. In one embodiment an algorithm that can create groups at any level of similarity from treating each cell as its own group to including all the cells in a single group is used. This embodiment may use the transformed and standardized biomarker expression level data as an input and groups the cells by proximity in multi-dimensional value space. Additional cell attributes that serve as input values may include relationships between the data for different biomarkers for a given cell and relationships between the occurrence of the same biomarker in different compartments of the same cell. For instance an additional cell attribute that the grouping algorithm considers could be the ratio between the expression level of two biomarkers in that cell or it could be the ratio of expression of a given biomarker in one compartment of that cell compared to the level of expression in another compartment of that cell. In this regard the level of similarity is just a shorthand way of referring to applying the grouping algorithm to yield a given number of groups.

The numerical tools used to implement the grouping algorithm may be any of those typically used to separate data into multiple groups. These range from the straightforward application of a set of rules or criteria to the more sophisticated routines of classical statistics including probability based analysis and learning algorithms such as neural networks.

The grouping algorithm may be applied in an unsupervised fashion meaning that no constraints beyond the level of similarity are applied with regard to how it creates groups or it may be applied in a partially supervised fashion, which means one or more constraints are applied. A typical constraint could be a requirement that all the cells possessing or lacking a particular attribute be included or excluded from one or more groups for that reason. For instance, the algorithm could be applied with the constraint that all cells expressing well above the mean amount of Glu1 be excluded from the groups it creates on the theory that these cells are suffering from hypoxia and therefore these cells do not provide representative information. The constraint may cause all the members of at least one group to share one or more attributes.

In an alternative approach the database may be interrogated with predefined profiles resulting in a fully supervised grouping. Thus one might extract a group of cells in which a biomarker for hypoxia, say Glu1 is expressed at levels well below the mean for all the examined tissue but that the marker for cell proliferation, say Ki67, is expressed at levels well above the mean for all the examined tissue.

Another interesting approach is to combine unsupervised, partially supervised and fully supervised grouping in an iterative manner. For instance one could identify a group of cells that have a threshold level of expression of certain proteins and then create subgroups of that group using unsupervised grouping based on a panel of biomarkers that might or might not include the original criteria proteins. In another case one could create subgroups of a group created by unsupervised grouping using partially or fully supervised grouping. In an other instance a group might be created by unsupervised grouping that is of particular interest and then a further application of the grouping routine could be used to identify other groups of cells that are similar to this group.

The cell attributes used to create the groups could include more than the patterns of biomarker expression. Additional attributes that could be considered include cell morphology and location in the tissue architecture such as proximity to a particular feature like a blood vessel.

The groupings created by the numerical tools or predefined profiles may be conveniently visualized by one or more overlays on images of the fields of view in which the analyzed cells appear. One approach is to take the images of one or more fields of view examined and overlay on such images symbols or colors representative of one or more of the groups such that the symbol or color representative of a given group is applied to all the cells in a given image that belong to that group. It is convenient to use an initial image or images in which cell boundaries are discernable but the signals from individual biomarkers are not displayed. In one embodiment the overlaid images are created by an electronic tool which allows the user to select the grouping iteration, i.e. the number of groups into which the cells have been classified and the number of those groups whose symbols are displayed. For instance, a user could select the grouping iteration that yielded seven groups and elect to display symbols for just two of those groups.

A numerical tool can be applied to the attribute data for all the cells belonging to a given group to determine whether there are any indications useful for diagnoses or prognoses of a disease or condition or for judging response to a treatment for a disease or condition. For instance, if samples are taken from tissue affected by a condition and tissues unaffected by the same condition all the cells belonging to a particular group can be examined to see if the cells in that group drawn from tissue which are affected by the condition display any attributes which distinguish them from the cells in that group drawn from tissue unaffected by that condition. One application could be to sample tissue affected by a neoplasm and normal tissue of the same type from the same subject or to sample tissue of the same type from subjects whose sampled tissue is cancerous and from subjects whose sampled tissue is normal. Then each group of cells created by the grouping algorithm can be examined to determine if there is any attribute that distinguishes cells from cancerous tissue from cells from normal tissue. In another instance historical tissue from a number of subjects with a cancerous condition whose survival rates since diagnoses are known can be examined by grouping cells and examining the attributes of the members of a group for an association with survival rates. In yet another application tissue samples could be taken from both subjects treated with a given therapy such as a drug and subjects not treated or treated with a placebo and examining all the cells in one or more groups created by the grouping algorithm for any attributes that distinguish the treated subjects from the control subjects. This approach can conveniently be applied to model animals such as mice implanted with neoplastic xenograft tissue from a human cancer.

The attributes examined may include not only the expression level and compartment location of the biomarkers evaluated but also interrelationships between these biomarkers and interrelationships between the expression levels of a given biomarker in different cellular compartments. For instance one could examine the ratio of expression levels of two biomarkers in a group of cells created by a grouping algorithm to see if the ratio could be associated with the presence of a condition or disease, the prognoses of the condition or disease or the treatment of the condition or disease with a particular therapy. One could similarly make use of the ratio of the levels of expression of a given biomarker between compartments of the same cell. In this instance it might be found that the cells from treated tissue in a given group had a different ratio of biomarker expression in the nucleus as compared to the cytoplasm for a given biomarker than the ratio for the cells for untreated tissue.

Another approach is to determine whether there is any association between the distribution of the groups and the diagnoses or prognoses of a condition or disease or the response of a condition or disease to a therapy. For instance it may be found that in tissue specimens from tissue that has gone neoplastic there are more cells in one or more of the groups than there are in healthy versions of the same tissue.

A particularly convenient statistical tool for examining the attributes of the cells in a group for indications useful for diagnoses, prognoses or treatment is “p-value” for association or probability that an observed association is the result of chance or random distribution.

### Example 1

A study was conducted on the effect of two cancer drugs and vehicle on a xenograft of human colon cancer tissue implanted in mice. Fixed, processed Xenograft tissue blocks were provided by Eli Lilly and Company (Indianapolis, Ind.) for further multiplexed analysis. A total of 39 HCT116 xenograft tumor bearing mice were treated three times a day for three days, and tumors were harvested four hours following last dose. Ten mice were treated with vehicle (DMSO), ten mice were treated with Enzastaurin at low dose (100 mpk), nine mice were treated with enzastaurin at high dose (200 mpk), Finally, 10 mice were treated with a dual PI3K/mTOR inhibitor at 30 mpk. Tumors were fixed in 10% neutral buffered formalin and processed for paraffin embedding and tissue sectioning. For the purpose of this study, tissues were sectioned from 15 animals: 5 from the vehicle treated group, 5 from the high dose Enzastaurin treated group and 5 from the dual PI3K/mTOR inhibitor treated group. The slides were baked at 65° C. for 1 hour. Paraffin was further removed from sample sections with Amresco's HistoChoice Clearing Agent for 15 minutes. The slides were then processed through a series of alcohol incubations of decreasing concentration of ethanol in water (100, 95. 70, 50%), twice at each concentration for 10 minutes, to hydrate the samples. The samples on the slides were then brought to saline conditions by incubation in PBS solution for 10 minutes. The crosslinked structures produced by formalin fixation were removed by a dual antigen retrieval method, where sample is placed in Sodium Citrate pH 6 in an pressure cooker for 25 minutes at high heat and allowed to cool to room temperature. Next, samples were transferred to a Tris/EDTA solution for another 25 minutes.

The slides prepared from the tumor tissue on each mouse were examined with a Zeiss Axiovision Z1 microscope equipped with high efficiency fluorochrome specific filter sets from Semrock for DAPI, Cy2, Cy3, and Cy5. Between 8 and 18 representative fields of interest were examined on each slide and selected for staining. Stage coordinates for each field of interest are marked using the Axiovision software and these coordinates are saved so they may be re-imaged after each staining round. A Piezzo X-Y automated stage allows the slide imaging system to repeatedly return to the same fields of interest. Fluorescence excitation is provided by a 300 W Xenon lamp source (Sutter Instrument). Images are captured with a Hammamatsu ORCA-IR CCD camera using Zeiss Axiovision software with initial exposure settings determined automatically within 75% saturation of pixel intensity.

Each slide was stained in succession with ten different fluorescently labeled antibodies, each one specific for one of the ten protein biomarkers listed in Table 1. The staining methodology was similar to that disclosed in U.S. Published Patent Application 2008/00118916 incorporated herein by reference. DAPI staining was performed at the first step and was re-stained if necessary in the subsequent steps. In general each slide was stained with 2 antibodies per round, labeled with Cy2, Cy3 or Cy5 fluorescent dyes and incubated overnight at 4C (alternatively a shorter time at room temperature is also possible). The slides were then mounted with media and coverslipped. The preselected regions of interest on each slide were imaged on the system as described above. Then each slide was removed and chemically bleached to destroy the signal from this set of fluorescent labels so that a further set of antibodies labeled with one or more of these fluorescent labels could be used. The chemical composition of the bleaching agent is described in U.S. Published Patent Application 2008/0118934, incorporated herein by reference. Then a second set of antibodies was used to stain a further two protein targets on each slide and so on until all the slides had been stained with antibodies specific to all ten protein biomarkers. Membrane regions were identified using Na—K-ATPase targeted antibodies, cytoplasm using S6 targeted antibodies and nuclei using DAPI stain. Additionally, other functional regions of interest within the tissue section were identified using Glu 1 for hypoxic regions and Ki67 for proliferating regions. One or more images were taken of each region of interest on each slide after each staining round. DAPI is not affected by the chemical bleaching agent and so the DAPI stained nuclei in each region of interest are also re-imaged in each imaging round (described in U.S. Published Patent Application 2008/0118934).

The pattern of said nuclei was used to place all the images of a given field of interest, a given stack, in registry. In particular, every image of a given field of interest captured the DAPI staining pattern showing the location of nuclei and the initial image was used as a reference to apply a rigid spatial transformation to the subsequent images so that the entire stack for that field of interest was in registry. The spatial transformation involved a global translation using a normalized correlation in a Fourrier domain followed by a rotational adjustment using a normalized mutual information metric starting from the intial translation obtained from the Fourrier transfrom. The registration transform was robust to intensity differences between the images in a given stack.

The auto-fluorescence, due to endogenous fluorophores in the tissue samples, was compensated for in accordance with the teachings of U.S. Patent Publication 2009/0141959 incorporated herein by reference. This involved capturing an image with an appropriate filter of each field of interest free of a given fluorescent stain and using it as a reference to remove the effects of auto-fluorescence from the image which records that fluorescent stain. As the same fluorescent dye was used multiple times the same reference image was used for correction each time a stain involving a given dye was used. In addition, another set of auto-fluorescence reference images were taken before the last three biomarkers were imaged and were used for auto-fluorescence correction for these last three biomarkers.

In the staining procedure each protein biomarker was associated with a particular fluorescent dye because the antibody used to detect that biomarker was coupled to a particular fluorescent dye. An image was then taken in the appropriate wavelength window for that fluorescent dye after the application of that antibody. Data was acquired for each compartment of each cell in each field of view representative of the level of expression of each protein biomarker for which a fluorescent stain had been applied. In particular, the intensity of the fluorescence at each pixel in each field of view was recorded for each protein biomarker.

Each pixel was associated with a particular subcellular compartment of a particular cell using software algorithms. The assignment of a given pixel to a given compartment of a given cell was based on an evaluation of the morphology of the tissue observed in the field of view and the stains applied to develop the nuclei, cytoplasm and cell membranes of the cells in the field of view. Although proprietary software was used, comparable if somewhat less accurate assignments could be obtained from commercially available segmentation software.

The pixels associated with certain punitive cells were then removed from the data set as a quality control measure. In particular, pixels associated with cells that did not contain a cytoplasm and one to two nuclei were eliminated, as were those associated with cells in regions in which the image quality was poor, cells not wholly within a given field of view and cells in the top 97^{th} percentile in terms of cell area. Between 25% and 30% of the pixels were eliminated by this procedure.

A database was now created in which each cell remaining after the quality control procedure was associated with certain attributes including a value reflective of the auto-fluorescence corrected fluorescence intensity for each of the ten protein biomarkers evaluated in total and in each of its subcellular compartments. In essence the fluorescence intensities of all the pixels associated with a given cell or a given compartment of a given cell associated with a given protein biomarker were summed The distribution of fluorescence intensities associated with each protein biomarker over the entire dataset was subjected to a Box Cox transformation to obtain an approximtely normal distribution. In most cases this led to the application of a power function of about 0.3. Then the transformed intensity values were standardized by determining the mean intensity value and the standard deviation of intensity values and subtracting the mean value from each actual value and dividing this difference by the standard deviation. Thus each cell and each compartment of each cell was provided with a post transformation standardized value for the fluorescent intensity of each of the ten protein biomarkers. This value was taken as representative of the level of expression of that biomarker in that cell or cellular compartment.

The database was then interrogated to create groups of cells with similar patterns of protein biomarker expression. In particular, a computer algorithm was used to iteratively group cells beginning in the first iteration with placing every cell in its own group and ending in a final iteration with placing all the cells in a single group. In each intermediate iteration cells that were more dissimilar in their patterns of protein biomarker expression were placed in the same group. The cell attributes used in this analysis were the standardized fluorescence level for each of the ten protein biomarkers and four ratios. The ratio inputs to this algorithm were for each cell the ratio of pS6 Serine 240 to S6 values, the ratio of pS6 Serine 235 to S6 values, the ratio of pAkt values in the cytoplasm to that in the membrane and the ratio of FOXO3a values in the nucleus to that in the membrane. For computational ease the grouping algorithm was applied to only 6000 representative cells, with an equal number of cells being selected from each field of view. Once biomarker profiles were created by the algorithm, these profiles were used to assign the remaining cells to appropriate groups, according to their degree of similarity in biomarker levels. For instance at the level of similarity that created three groups, three different biomarker profiles were created. The cells not part of the original 6000 cell sample were assigned to one of the three groups whose biomarker profile they most closely matched.

The expression levels of each protein biomarker, as well as each of the four ratios, was analyzed for its associations with cells belonging to the control mice, the mice treated with Enzastaurin or the mice treated with the dual PI3K/mTOR inhibitor, at each level of similarity. For biomarker similarity levels yielding a single group and three groups, only an association involving the single protein biomarker S6 was found. In the former case a higher level of expression of S6 was correlated to treatment with either of the anti-cancer drugs while in the latter case a similar association was found for just the third group of cells. For biomarker similarity levels yielding five and seven cell groups, a number of associations were found. The biomarker profiles for the three, five and seven cell groups are shown in Tables 2, 3 and 4. The associations found for the latter two biomarker similarity levels are shown in Tables 5 and 6. In profile Tables 2, 3 and 4 the occurrence of each attribute for each profile is compared to the mean value of that attribute for all the cells in the dataset. The indication “+/−” means that the value for that profile is essentially the same as the average value of that attribute for all the cells in the dataset. The indication “++” means a value of one standard deviation or more above the mean value and the indication “−−” means a value one standard deviation or more below the mean. The indications “+” and “−” mean a value between the mean and one standard deviation above or below the mean, respectively. In tables 5 and 6 the associations are reported in “p” values that indicate the probability that the observed difference between the compared groups could have occurred by chance.

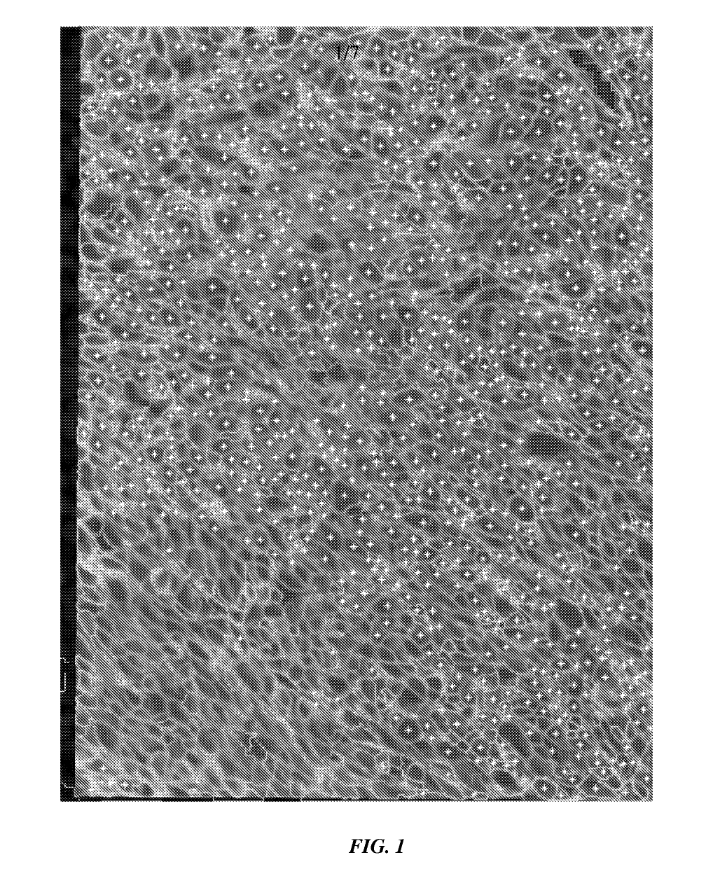

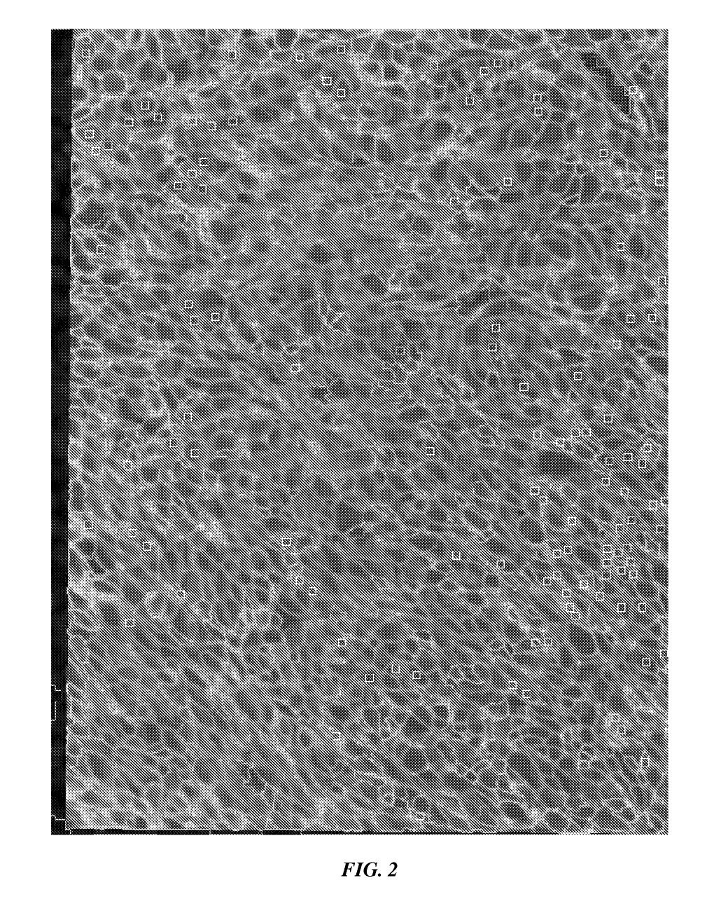

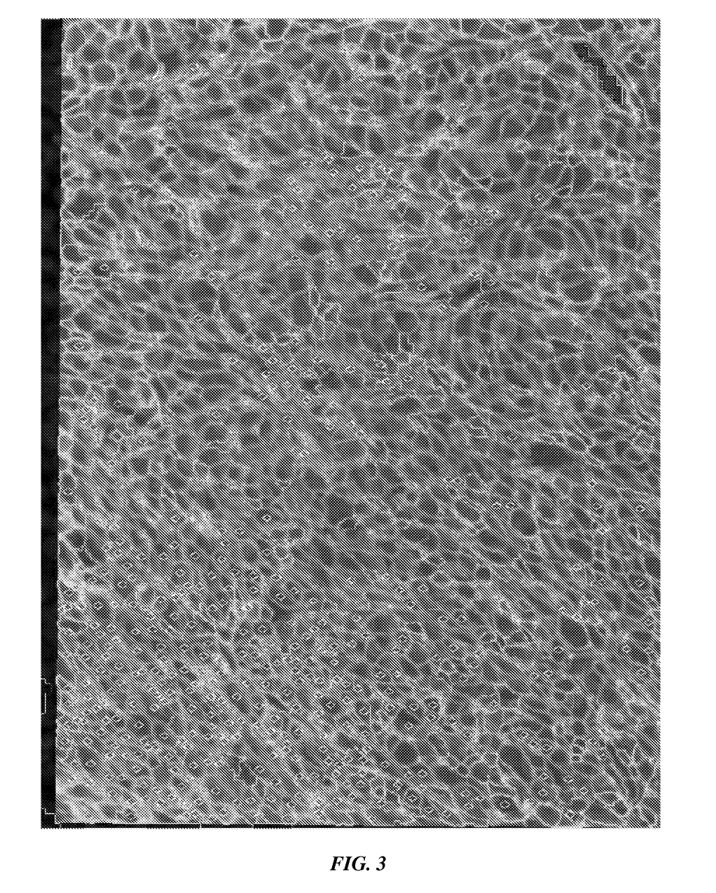

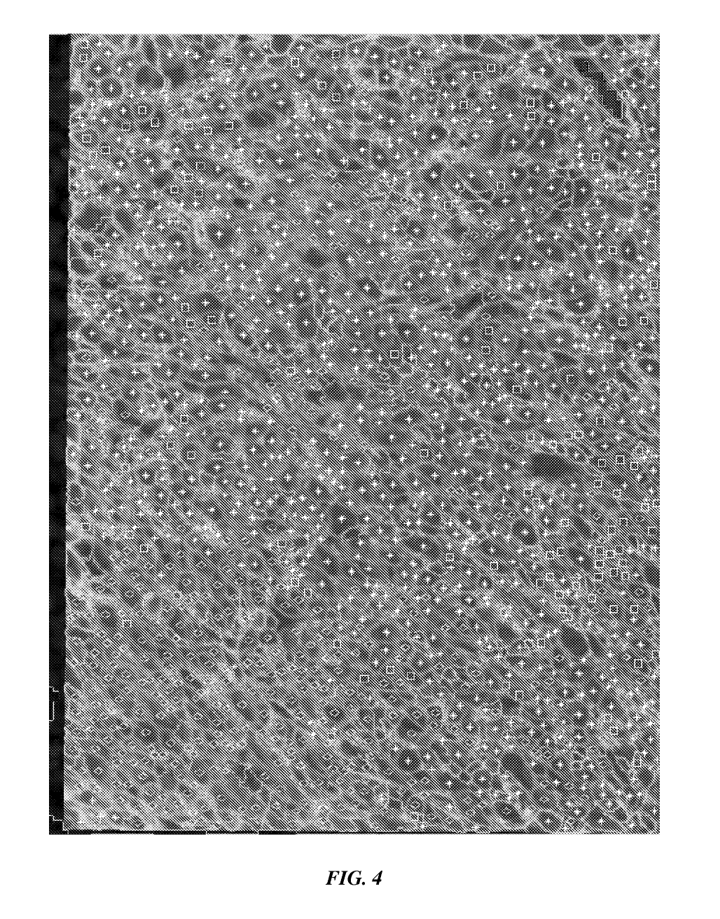

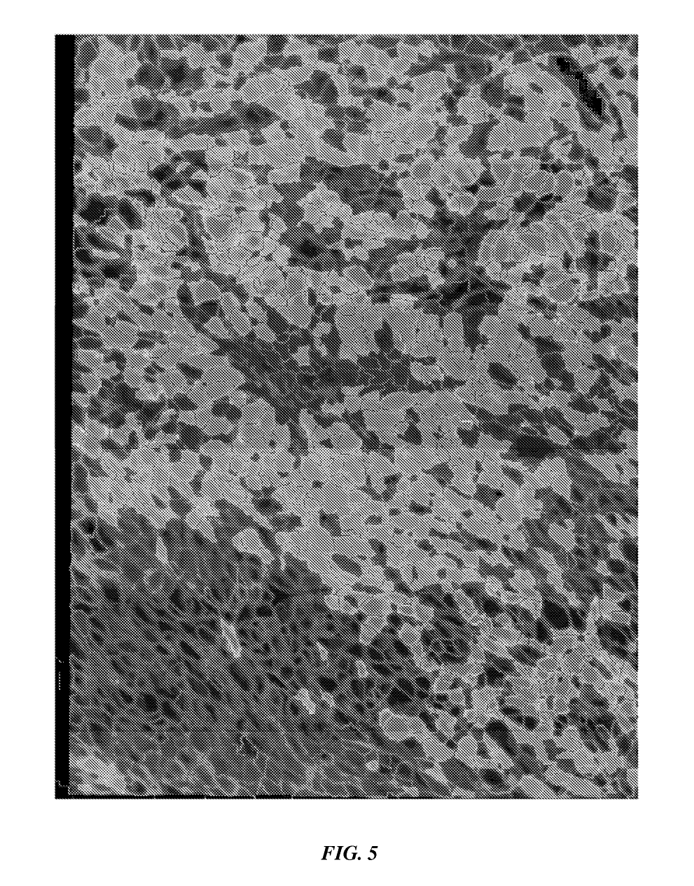

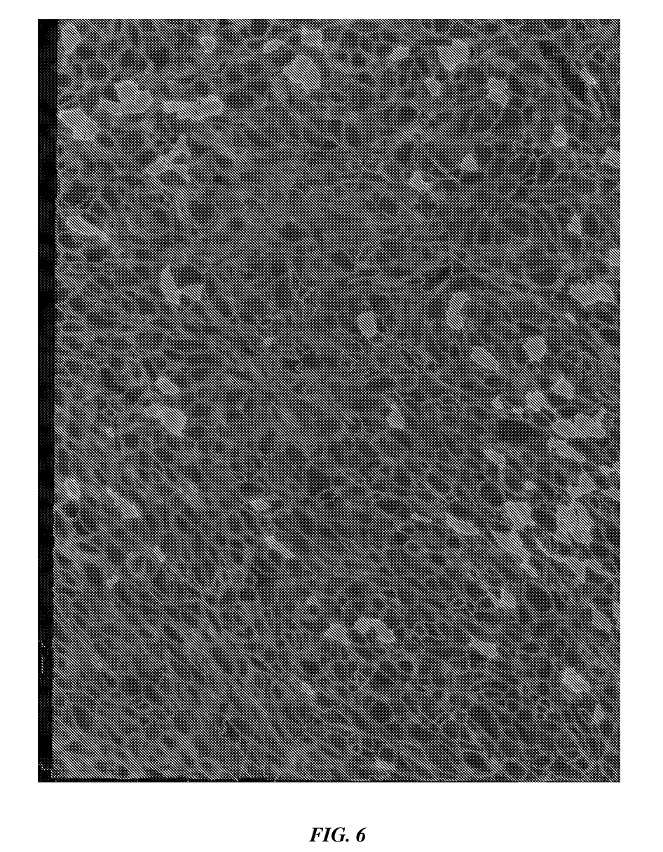

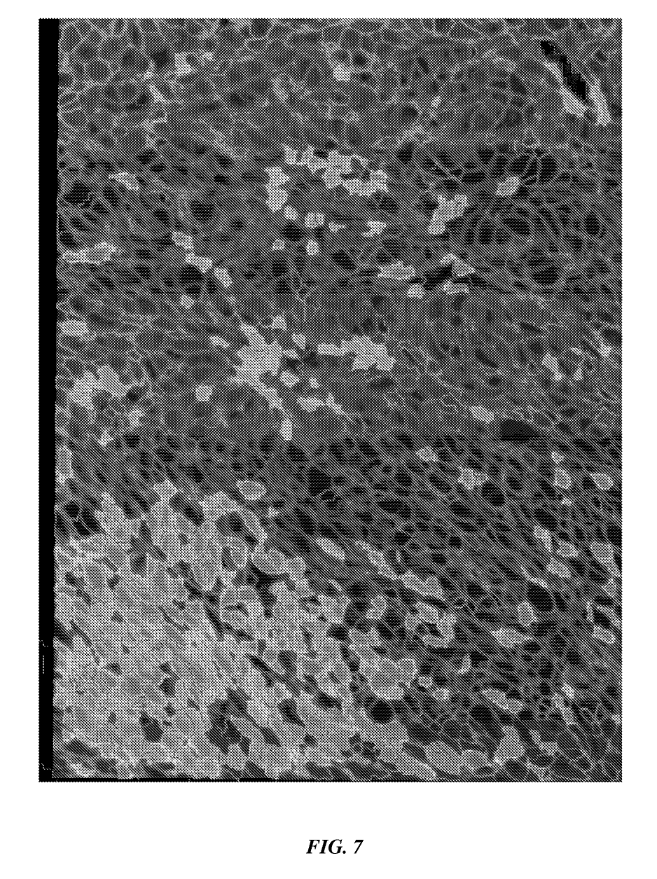

FIGS. 1-7 illustrate various ways of displaying the groups created in accordance with Table 2. FIGS. 1, 2 and 3 illustrate each of the three groups indicated by a particular symbol on an image containing cells that were examined to create the groups. FIG. 4 illustrates visualizing all three groups simultaneously using a different symbol for each. FIGS. 5, 6 and 7 illustrate an alternative technique of visualizing each of the three groups by shading in the individual cells that are members of each group.

TABLE 1
Cell Attributes Examined

| Attribute | Biomarker |
|---|---|
| Expression Level | FOXO3a |
| Expression Level | Glu1 |
| Expression Level | Ki67 |
| Expression Level | S6 |
| Expression Level | pAKt |
| Expression Level | pCREB |
| Expression Level | pCAD |
| Expression Level | pGSK3beta |
| Expression Level | pS6 Serine 235 |
| Expression Level | pS6Serine 240 |
| Ratio of Expression Levels | pS6 Serine 240/S6 |
| Ratio of Expression Levels | pS6 Serine 235/S6 |
| Ratio of Expression Levels | pAkt (cyto/mem) |
| Ratio of Expression Levels | FOXO3a (nuc/mem) |

TABLE 2
Profiles for Three Group Analysis

| Cell Attribute | Group 1 | Group 2 | Group 3 |
|---|---|---|---|
| FOXO3a Level | − | − | + |
| Glu1 Level | − | + | + |
| Ki67 Level | + | − | + |
| S6 Level | − | − | + |
| pAKt Level | − | + | + |
| pCREB Level | +/− | −− | + |
| pCAD Level | − | − | + |
| pGSK3beta Level | − | − | + |
| pS6 Serine 235 Level | − | + | − |
| pS6 Serine 240 Level | − | − | + |
| pS6 Serine 240/S6 Ratio | + | − | +/− |
| pS6 Serine 235/S6 Ratio | + | − | − |
| pAkt (cyto/mem) Ratio | + | − | +/− |
| FOXO3a | − | − | + |
| (nuc/mem) Ratio | | | |

TABLE 3
Profiles for Five Group Analysis

| Cell Attribute | Group 1 | Group 2 | Group 3 | Group 4 | Group 5 |
|---|---|---|---|---|---|
| FOXO3a Level | −− | − | − | + | + |
| Glu1 Level | −− | − | + | + | +/− |
| Ki67 Level | − | +/− | − | − | + |
| S6 Level | −− | − | − | + | + |
| pAKt Level | −− | − | − | + | + |
| pCREB Level | − | + | −− | + | + |
| pCAD Level | − | − | − | + | + |
| pGSK3beta Level | − | − | − | + | + |
| pS6 Serine 235 Level | − | +/− | ++ | + | − |
| pS6 Serine 240 Level | +/− | +/− | −− | +/− | + |
| pS6 Serine 240/S6 Ratio | + | − | −− | − | + |
| pS6 Serine 235/S6 Ratio | + | + | − | − | + |
| pAkt (cyto/mem) Ratio | + | + | − | +/− | − |
| FOXO3a | − | +/− | − | − | + |
| (nuc/mem) Ratio | | | | | |

TABLE 4
Profiles for Seven Group Analysis

| Cell Attribute | Group 1 | Group 2 | Group 3 | Group 4 | Group 5 | Group 6 | Group 7 |
|---|---|---|---|---|---|---|---|
| FOXO3a Level | −− | − | − | + | + | + | +/− |
| Glu1 Level | −− | − | − | + | + | + | − |
| Ki67 Level | − | − | − | +/− | −− | − | + |
| S6 Level | −− | − | − | + | − | + | + |
| pAKt Level | −− | − | −− | + | +/− | + | + |
| pCREB Level | − | + | −− | − | −− | + | + |
| pCAD Level | − | − | − | + | − | + | + |
| pGSK3beta Level | −− | − | − | +/− | + | ++ | + |
| PS6 Serine 235 Level | − | − | ++ | + | + | +/− | − |
| PS6 Serine 240 Level | + | − | −− | − | − | + | ++ |
| pS6 Serine 240/S6 Ratio | + | + | − | − | + | + | + |
| pS6 Serine 235/S6 Ratio | + | + | − | − | +/− | − | + |
| pAkt (cyto/mem) Ratio | + | + | + | − | +/− | + | − |
| FOXO3a | −− | +/− | − | + | − | + | + |
| (nuc/mem) Ratio | | | | | | | |

TABLE 5
Associations Between Treatment and Attribute for Five Group Analysis

| Cell Attribute | Group 1 | Group 2 | Group 3 | Group 4 | Group 5 |
|---|---|---|---|---|---|
| | Control vs. ENZA | | | | |
| FOXO3a Level | | | | | |
| Glu1 Level | | | | | |
| Ki67 Level | p = 0.014 (−) | | | | |
| S6 Level | | | | | p = 0.034 (+) |
| pAKt Level | | | | | |
| pCREB Level | | | | | |
| pCAD Level | | | | | |
| pGSK3beta Level | | | | | |
| pS6 Serine 235 Level | | | | | |
| pS6 Serine 240 Level | | | | | |
| pS6 Serine 240/S6 Ratio | | | | | |
| pS6 Serine 235/S6 Ratio | | | | | |
| pAkt (cyto/mem) Ratio | | | | | |
| FOXO3a | | | | | |
| (nuc/mem) Ratio | | | | | |
| | Control vs. the dual PI3K/mTOR inhibitor | | | | |
| FOXO3a Level | p = 0.034 (−) | | | | |
| Glu1 Level | | | | | |
| Ki67 Level | | | | | |
| S6 Level | | | | p = 0.032 (+) | p = 0.023 (+) |
| pAKt Level | | | | | |
| pCREB Level | | | | | |
| pCAD Level | | | | | |
| pGSK3beta Level | | | | | |
| PS6 Serine 235 Level | p = 0.033 (−) | | | | |
| PS6 Serine 240 Level | p = 0.049 (−) | | | | p = 0.021 (−) |
| pS6 Serine 240/S6 Ratio | | | | p = 0.010 (−) | |
| pS6 Serine 235/S6 Ratio | | | | | |
| pAkt (cyto/mem) Ratio | | | | p = 0.019 (−) | |
| FOXO3a | | | | | |
| (nuc/mem) Ratio | | | | | |

TABLE 6
Associations Between Treatment and Attribute for Seven Group Analysis

| Cell Attribute | Group 1 | Group 2 | Group 3 | Group 4 | Group 5 | Group 6 | Group 7 |
|---|---|---|---|---|---|---|---|
| | Control vs. ENZA | | | | | | |
| FOXO3a Level | | | | | | | |
| Glu1 Level | | | | | | | |
| Ki67 Level | p = 0.015 (−) | | | | | | |
| S6 Level | | | | p = 0.023 (+) | | | p = 0.017 (+) |
| pAKt Level | | | | | | | |
| pCREB Level | | | | p = 0.047 (+) | | | |
| pCAD Level | | | | | | | |
| pGSK3beta Level | | | | | | | |
| PS6 Serine 235 Level | | | | | | | |
| PS6 Serine 240 Level | | | | | | | |
| pS6 Serine 240/S6 Ratio | | | | | | | |
| pS6 Serine 235/S6 Ratio | | | | | | | |
| pAkt (cyto/mem) Ratio | | | | | p = 0.018 (−) | | |
| FOXO3a | | | | | | | |
| (nuc/mem) Ratio | | | | | | | |
| | Control vs. the dual PI3K/mTOR inhibitor | | | | | | |
| FOXO3a Level | p = 0.039 (−) | | | | | | |
| Glu1 Level | | | | | | | |
| Ki67 Level | | | | | p < 0.001 (−) | | |
| S6 Level | | | | | | | |
| pAKt Level | | | | | | | |
| pCREB Level | | | | p = 0.037 (+) | | | |
| pCAD Level | | | | | | | |
| pGSK3beta Level | | | | | | | |
| pS6 Serine 235 Level | | | | | | | |
| pS6 Serine 240 Level | p = 0.030 (−) | | | | | | p = 0.022 (−) |
| pS6 Serine 240/S6 Ratio | p = 0.033 (−) | | | | p = 0.012 (−) | p = 0.004 (−) | p = 0.013 (−) |
| pS6 Serine 235/S6 Ratio | | | | | | | |
| pAkt (cyto/mem Ratio) | | | | | p = 0.022 (−) | | |
| FOXO3a | | | | | | | |
| (nuc/mem) Ratio | | | | | | | |

While only certain features of the invention have been illustrated and described herein, many modifications and changes will occur to those skilled in the art. It is, therefore, to be understood that the appended claims are intended to cover all such modifications and changes as fall within the true spirit of the invention.

1. A process for acquiring data for analysis of the patterns of expression of multiple biomarkers in cells in their tissue of origin comprising:
a. Acquiring one or more digital images of a field of view of a microscope of a tissue specimen treated to reveal the level of expression of multiple biomarkers;
b. Segmenting the images into individual cells or the cellular compartments of individual cells;
c. Determining the level of expression of multiple biomarkers within in the individual cells or the cellular compartments of individual cells; and
d. Storing the data acquired in a dataset such that the association of a given set of values with a given cell or the compartments of a given cell are preserved.

2. The process of claim 1 wherein the data is stored such that the dataset can be interrogated to yield groups of cells with similar patterns of biomarker expression.

3. The process of claim 1 wherein multiple images are acquired of a given field of view and the each image contains a feature used to place the multiple images in registry with each other.

4. The process of claim 3 wherein the registry feature is the pattern of nuclei in each image.

5. The process of claim 1 wherein said tissue specimen is subjected to multiple treatments to develop signals representative of the expression levels of the multiple biomarkers.

6. The process of claim 5 wherein said tissue specimen is iteratively stained with a set of one or more probes, each specific for a given biomarker and carrying a fluorescent label different from the other probes in that set, subjected to a bleaching treatment which inactivates the fluorescent labels associated with said probes and then stained with another set of one or more additional fluorescently labeled probes, each specific to a biomarker not yet stained and carrying a fluorescent label different from the other probes in that set and wherein a digital image is taken after each staining treatment.

7. The process of claim 1 wherein pixels of the digital images of said field of view is are associated with a cellular compartment of an individual cell.

8. The process of claim 1 wherein a step in interrogating the dataset is a quality control step in which data from certain cells is not further considered in interrogating said dataset.

9. The process of claim 1 wherein a grouping algorithm is applied to said dataset that groups together cells with similar patterns of biomarker expression using a numerical tool.

10. The process of claim 1 wherein data is obtained from the same tissue of numerous subjects and the subjects are placed in groups based on how similar each subject's expression of said biomarkers is to other members of the group and wherein the grouping analysis is based on biomarker expression data summarized at the cell level or cell compartment level.

11. A process for analyzing data derived from digital images of pathological specimens which is representative of the level of expression of multiple biomarkers in the cells included in said images comprising:
a. Acquiring data from each digital image to be included in said analysis which is representative of the level of expression of multiple biomarkers in individual cells included in said image; and
b. Comparing the data representative of level of expression of multiple biomarkers in a given cell to data representative of level of expression of the same biomarkers in the other cells included in the analysis and creating profiles of biomarker expression which group together cells with similar biomarker expression patterns using a computer algorithm wherein such similarity is determined by a numerical analysis which uses the level of expression of each biomarker as a continuous variable.

12. The process of claim 11 wherein the numerical analysis is a rule based analysis, a classical statistical analysis or learning algorithm.

13. The process of claim 12 wherein a probability based classical statistical analysis is used.

14. The process of claim 12 wherein a neural network based learning algorithm is used.

15. A process for creating profiles of cellular expression of multiple biomarkers comprising:
a. Acquiring one or more digital images of numerous cells in situ in one or more tissue specimens that have been treated to yield signals representative of the level of certain biomarkers present in the cells of said one or more tissue specimens;
b. Analyzing said digital images to determine the level of expression of said biomarkers in numerous cells included in said digital images to generate data for each such cell which is representative of its level of expression of said biomarkers;
c. Comparing the data representative of level of expression of multiple biomarkers in a given cell to data representative of level of expression of the same biomarkers in the other cells included in the analysis and creating profiles of biomarker expression which group together cells with similar biomarker expression patterns using a computer algorithm wherein such similarity is determined by a numerical analysis which uses the level of expression of each biomarker as a continuous variable.

16. The process of claim 15 wherein multiple digital images are made of each tissue specimen and between images the specimen is treated to yield a signal representative of the level of expression of a biomarker not previously capable of yielding a signal.

17. The process of claim 16 wherein said multiple images of the same tissue specimen are kept in registry with each other by a marker which is common to all of said images.

18. The process of claim 17 wherein said marker is a marker that identifies a cellular compartment.

19. The process of claim 15 wherein the analysis includes a determination of the level of expression of said biomarkers in at least two compartments of the cells analyzed and the similarity analysis takes account of such compartment data.

20. The process of claim 19 wherein the compartments considered are the nucleus, cytoplasm and membrane.

21. The process of claim 19 wherein for at least one biomarker a ratio is created between the levels of expression of that biomarker in at least two compartments in each cell included in the analysis and said ratio data is utilized by the computer algorithm used to group cells.

22. The process of claim 15 wherein the biomarker is a protein, a DNA sequence or an RNA sequence.

23. A process for creating profiles of cellular expression of multiple biomarkers comprising:
a. Acquiring one or more digital images of each of numerous cells in situ in one or more tissue specimens that have been treated to yield signals representative of the level of certain biomarkers present in the cell of said one or more tissue specimens;
b. Analyzing said digital images to determine the level of expression of said biomarkers in numerous cells included in said digital images to generate data for each such cell which is representative of its level of expression of said biomarkers;
c. Creating a dataset in which are stored the level of expression of said biomarkers in each individual cell for which such data is created;
d. Interrogating said dataset for groups of cells whose members have a similar pattern of expression of said biomarkers using a computer algorithm wherein such similarity is determined by a numerical analysis that uses the level of expression of each biomarker as at least a semi-continuous variable; and
e. Determining a profile of cellular expression for each group created which is based on a central value for each cellular attribute considered by said algorithm.

24. The process of claim 23 wherein said numerical analysis that uses the level of expression of each biomarker as a continuous variable.

25. The process of claim 23 wherein the level of expression of each biomarker is assigned to one of at least three groups and this grouping rather than the actual value of the level of expression is used in said numerical analysis.

26. The process of claim 23 wherein said central value is the mean or median of each cellular attribute considered by said algorithm.

27. A process for analyzing data representative of the level of expression in individual cells of multiple biomarkers comprising:
a. Acquiring data on the level of expression of each of the biomarkers of interest for each of the cells of interest;
b. Creating a dataset in which are stored the levels of expression of said biomarkers in said individual cells; and
c. Using a computer algorithm to create groups of cells whose members have a similar pattern of expression of said biomarkers wherein such similarity is determined by a numerical analysis that uses the level of expression of each biomarker as a variable.

28. The process of claim 27 wherein the data on the level of expression in individual cells is acquired from digital images of the cells in situ in the tissue in which they occur.

29. The process of claim 28 wherein said images are acquired from the tissue of multiple subjects.

30. The process of claim 29 wherein the pattern of occurrence of cells belonging to a particular group is compared for tissue belonging to different subjects.

31. The process of claim 30 wherein the difference in patterns is used to diagnose a pathological condition.

32. The process of claim 30 wherein the difference in patterns is used to form a prognosis of a pathological condition.

33. The process of claim 31 or 32 wherein the pathological condition is a neoplasm.

34. The process of claim 27 wherein for each cell of interest the data includes the level of expression in each of the cellular compartments of nucleus, cytoplasm and membrane of that cell for each biomarker of interest and this data is utilized by the computer algorithm that creates the groups.

35. The process of claim 27 wherein constraints are imposed computer algorithm used to create groups of cells that require all the cells in one or more groups to posses one or more attributes.

36. The process of claim 27 wherein a group of cells is divided into subgroups using one or more external constraints.

37. The process of claim 27 wherein the initial grouping or a subsequent subgrouping of said cells utlilizes one or more cell attributes in addition to the pattern of biomarker expression.

38. The process of claim 37 wherein said additional one or more cell attributes involve cell morphology, location in the tissue architecture or both.

39. The process of claim 27 wherein the computer algorithm considers not only the level of expression of each biomarker of interest but also at least one interrelationship between the levels of expression for two of said biomarkers.

40. The process of claim 34 wherein the computer algorithm considers not only the level of expression of each biomarker of interest in each said cellular compartment but also the interrelationship between the levels of expression of at least one biomarker in at least two of said cellular compartments.

41. The process of claim 27 wherein data is obtained from the same tissue of numerous subjects and the subjects are placed in groups based on how similar each subject's distribution of cell groups is to other members of the group.

42. A process for identifying one or more biomarkers whose levels of expression in a predefined group of cells is indicative of the presence, prognoses or response to treatment of a condition or disease comprising:
a. Obtaining data on the level of expression for each of a number biomarkers in each individual cell of a sampling of cells
1. from one or more tissue specimens from a subject or subjects having a given condition or disease and from one or more tissue specimens from a subject or subjects free of a given condition or disease; or
2. from historical tissue specimens from subjects having a condition or disease wherein some of said subjects had different clinical outcomes than other of said subjects; or
3. from tissue specimens from a subjects having a given condition or disease, wherein some of said subjects have been subjected to a therapy for said condition or disease and others have not been so subjected; or
4. from tissue specimens from subjects having a given condition or disease wherein some of the subjects have been subjected to one level of a therapy for the condition or disease and other of the subjects have been subjected to a different level of therapy;
b. Creating a dataset in which are stored the levels of expression of said biomarkers in said individual cells;
c. Applying a computer algorithm to said dataset to create groups of cells whose members have a similar pattern of expression of said biomarkers wherein such similarity is determined by a numerical analysis that uses the level of expression of each biomarker as a variable; and
d. Examining the level of expression of each of the biomarkers in a given group for an association to the presence, prognoses or response to treatment of said condition or disease.

43. The process of claim 42 wherein level of expression of each of the biomarkers in a given group is examined for an association to the presence, prognoses or response to treatment of said condition or disease.

44. A process for displaying one or more groups of cells having similar patterns of expression of certain biomarkers comprising:
a. Acquiring data on the level of expression of each of said biomarkers for each of the cells examined in portions of one or more tissue specimens;
b. Creating a dataset in which are stored the levels of expression of said biomarkers in said individual cells;
c. Using a computer algorithm to create groups of cells whose members have a similar pattern of expression of said biomarkers wherein such similarity is determined by a numerical analysis that uses the level of expression of each biomarker as a variable; and
d. Creating an image of one of the portions originally examined of one of the tissue specimens in which the cells in that portion belonging to at least one said group are given a visible designation that they belong to the same group.

45. The process of claim 44 wherein multiple groups of cells are each given a different visible designation that is overlaid on the image of the portion of the tissue specimen.
